SEQUENCE LISTING (31 sequences)

SEQ ID NO: 1 - DNA, 50 bp, Artificial Sequence
  synthetic primer for the site-directed mutagenesis
cctccccctgaacctgaaacaagatctgaatgcaattgttgttgttaacg

SEQ ID NO: 2 - DNA, 51 bp, Artificial Sequence
  synthetic primer for the site-directed mutagenesis
cgttaacaacaacaattgcattcaggatcttgtttcaggttcagggggagg

SEQ ID NO: 3 - DNA, 16 bp, Artificial Sequence
  synthetic primer, RT-telo-4
ctcatatattcagtat

SEQ ID NO: 4 - DNA, 15 bp, Artificial Sequence
  synthetic primer, RT-telo-3
ctggacactcgctca

SEQ ID NO: 5 - DNA, 15 bp, Artificial Sequence
  synthetic primer, RT-telo-2
tcagccggacatgca

SEQ ID NO: 6 - DNA, 15 bp, Artificial Sequence
  synthetic primer, RT-telo-1
tcactcaggcctcag

SEQ ID NO: 7 - DNA, 17 bp, Artificial Sequence
  synthetic primer, RCR-telo-10
gctggtgtctgctctcg

SEQ ID NO: 8 - DNA, 25 bp, Artificial Sequence
  synthetic primer, RCR-telo-11
ctgcagcaggaggatcttgtagatg

SEQ ID NO: 9 - DNA, 21 bp, Artificial Sequence
  synthetic primer, RCR-telo-6
gcaggtgaacagcctccagac

SEQ ID NO: 10 - DNA, 24 bp, Artificial Sequence
  synthetic primer, RCR-telo-R13
cacaggctgcagagcagcgtggag

SEQ ID NO: 11 - DNA, 25 bp, Artificial Sequence
  synthetic primer, RCR-telo-F12
gtcctacgtccagtgccaggggatc

SEQ ID NO: 12 - DNA, 25 bp, Artificial Sequence
  synthetic primer, RCR-telo-R15
gagcacgctgaaccagtgccttcac

SEQ ID NO: 13 - DNA, 24 bp, Artificial Sequence
  synthetic primer, RCR-telo-F14
agagggccgagcgtctcacctcga

SEQ ID NO: 14 - DNA, 26 bp, Artificial Sequence
  synthetic primer, RCR-telo-R17
cgctcatcttccacgtcagctcctgc

SEQ ID NO: 15 - DNA, 24 bp, Artificial Sequence
  synthetic primer, RCR-telo-F16
ctcaggaacaccaagaagttcatc

SEQ ID NO: 16 - DNA, 25 bp, Artificial Sequence
  synthetic primer, RCR-telo-R19
cctggcatccagggcctggaaccca

SEQ ID NO: 17 - DNA, 27 bp, Artificial Sequence
  synthetic primer, RCR-telo-F18
tccctactcagctctctgaggcccagc

SEQ ID NO: 18 - DNA, 29 bp, Artificial Sequence
  synthetic primer, RCR-telo-F20
ttgctggtggctcccagctgcgcctagga

SEQ ID NO: 19 - DNA, 25 bp, Artificial Sequence
  synthetic primer, RCR-telo-R21
agtggcagcgccgagctggtacagc

SEQ ID NO: 20 - DNA, 24 bp, Artificial Sequence
  synthetic primer, RCR-telo-7
atgccgcgcgctccccgctgccag

SEQ ID NO: 21 - DNA, 49 bp, Artificial Sequence
  synthetic primer for site-directed PCR
aagcttgatatcgaattcgggtaccatgccgcgcgctccccgctcccgg

SEQ ID NO: 22 - DNA, 51 bp, Artificial Sequence
  synthetic primer for site-directed PCR
gacttcaagaccatcctggacaccggtccacccgcccacagccaggccgag

SEQ ID NO: 23 - DNA, 51 bp, Artificial Sequence
  synthetic primer for site-directed PCR
caccattgagtttaaacccgcaagcttgcctcgactgtgccttctagttgc

SEQ ID NO: 24 - DNA, 51 bp, Artificial Sequence
  synthetic primer for site-directed PCR
tacatttcttacatctatgcgaagctttggggatccgagctcgagatctgc

SEQ ID NO: 25 - DNA, 57 bp, Artificial Sequence
  synthetic primer for site-directed PCR
acacctccccctgaacctgaaacaaccggtgaatgcaattgttgttgttaacgggga

SEQ ID NO: 26 - DNA, 57 bp, Artificial Sequence
  synthetic primer for site-directed PCR
ggagacccaagctggctagttaagagatctatgacctctcgccgctccgtgaagtcg

SEQ ID NO: 27 - DNA, 20 bp, Artificial Sequence
  synthetic oligonucleotide
gatccagatctgtttaaacg

SEQ ID NO: 28 - DNA, 20 bp, Artificial Sequence
  synthetic oligonucleotide
gatccgtttaaacagatctg

SEQ ID NO: 29 - DNA, 3075 bp, Artificial Sequence
  VP22-TAg gene fusion
atgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcttccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgtctaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggctgtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgagggtaccgagctcggatcccgcgaccgctcgaccagctttgcaaagatggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacataa

SEQ ID NO: 30 - DNA, 3036 bp, Artificial Sequence
  VP22-TAg gene fusion
atggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacaagatctaccatgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcatccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgactaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggcggtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgag

SEQ ID NO: 31 - DNA, 3544 bp, Artificial Sequence
  Telomerase-Fragments in pcrscript-Telomerase
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcaggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcggggctgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagca

(19) United States
(12) Patent Application Publication
(10) Pub. No.: US 2007/0237754 A1
(43) Pub. Date: Oct. 11, 2007

(54) Transient immortalization

(76) Inventors: Jan-Heiner Kupper, Kusterdingen (DE); Ralph Meyer, Waldbockelheim (DE); Mirella Meyer-Ficca, Waldbockelheim (DE); Anne Kuhn, Tubingen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/804,931

(22) Filed: May 21, 2007

Related U.S. Application Data
(63) Continuation-in-part of application No. 10/492,763, filed on May 20, 2004, now abandoned.

(30) Foreign Application Priority Data
Oct. 18, 2001 (DE) 101 52 972.4

(51) Int. Cl. A61K 48/00; A01N 1/00; C07K 14/00; C12N 5/02; C12N 9/00

(52) U.S. Cl. 424/93.21; 435/1.1; 435/183; 435/325; 435/375; 530/350

(57) ABSTRACT

The invention relates to a method for transiently immortalizing cells according to which immortalization proteins are introduced into the cells from outside. The invention also relates to a method for producing cells according to which organ-related cells are transiently immortalized by the exogenous supply of immortalization proteins and are remortalized after their expansion. The invention further relates to the cells produced according to the inventive method, to the use of said cells for producing a transplant and to the immortalization proteins used in the method.

### CROSS-REFERENCE TO RELATED APPLICATIONS

This application is a Continuation-in-Part of U.S. application Ser. No. 10/492,763, filed May 20, 2004, which is a US National Phase of International Patent Application No.: PCT/EP02/11200, filed Oct. 7, 2002, designating the US and published not in English on May 1, 2003 as WO 03/035884, which claims the benefit of German Patent Application No.: 101 52 972.4, filed Oct. 18, 2001.

### BACKGROUND OF THE INVENTION

1. Field of the Invention

The present invention is concerned with methods for obtaining cells which can be transplanted, for example into an organ. In general terms, the present invention relates to degenerative diseases which are associated with the destruction of defined cell populations and to transplants and drugs for treating degenerative diseases of this nature.

2. Description of the Related Art

Particularly as a result of the changing age pyramid, chronically degenerative diseases which are difficult or not yet possible to treat are increasing in the industrialized countries. These diseases include, inter alia, cardiac muscle diseases, neurodegenerative diseases, bone diseases and liver diseases which are characterized by the loss of relevant cell populations.

In cardiac infarction, for example, heart muscle cells are irreversibly destroyed, while the islet cells of the pancreas are destroyed in insulin-dependent diabetes mellitus, as a consequence of an autoimmune disease, and the dopamine-producing cells in the substantia nigra are destroyed in Parkinson's disease, to mention only a few of the most important diseases.

In virtually no instance are the natural processes of regeneration able to replace these functionally important cells. For this reason, a great advance in the treatment of degenerative diseases is seen in growing these organ-related cells outside the body and, after having propagated them appropriately, transplanting them into the damaged organs. If the cells are endogenous to the body, it is probable that the regeneration of the organs will be long-lasting since no tissue rejection reactions will take place.

These organ-related cells can nowadays be obtained from embryonic and adult stem cells. For example, it is possible to obtain cardiac muscle cells from mesenchymal stroma cells of the bone marrow. However, these cells are only able to divide to a limited extent and the number of cell divisions is not sufficient to obtain the requisite number of organ-related cells. For this reason, efforts are being made to immortalize these cells in order to be able to produce them in unlimited quantity. It is possible to achieve immortalization by introducing the gene function for at least the catalytic subunit of human telomerase (hTRT) into primary cells. In many cases, other gene functions are also needed in order to overcome the cell cycle arrest of primary cells so as to enable these cells to begin dividing in the first place. These gene functions usually have transforming or oncogenic properties. The SV40 large tumor antigen is a prototype of these gene functions.

It has been known for a long time that primary cell cultures have only a limited capacity for cell division. In 1961, Leonard Hayflick of the Wistar Institute discovered that, while fibroblasts from newborn infants can make 80-90 cell divisions, those from 70 year-old individuals still only divided 20-30 times. After these numbers of divisions, the cells go into senescence. The age of the donor determines the replicative capacity.

It is nowadays known that this replicative capacity is determined by the length of the telomeres, i.e. the ends of the chromosome. In normal cells, the telomeres shorten in conjunction with each cell division. The telomeres consist of repeats of a hexamer sequence, which is TTAGGG in mammals, and are approximately 12 kb in length in the newborn human. This loss occurs in most somatic cells. Germ line cells possess an enzyme function which is able to redress this replication loss. This enzyme function, which is termed telomerase, was discovered for the first time by Elizabeth Blackburn and Carol Creider in the unicellular organism Tetrahymena, which is a ciliate. Telomerase is a ribonucleoprotein. The RNA moiety, which is encoded by a separate gene, contains the template sequence for the telomerase reaction. The gene for this template RNA has by now been cloned from many organisms, including man. The other telomerase factors have also by now been cloned from a variety of species. Telomerase additionally consists of a P80 protein, which binds the RNA template, and a P95 protein, which provides the polymerase function. Telomerase is consequently a special reverse transcriptase which uses a bound RNA to generate a fragment of DNA at the chromosome ends.

In this connection, telomere-binding proteins ensure that the extension of the chromosome ends takes place in a regulated manner. The gene for the 60 kDa telomere repeat factor TRF has been cloned from human cells. The protein possesses a DNA-binding domain which exhibits homology with the MYB oncoprotein and which is also found in the homologous yeast protein RAP1. The binding of TRF and other proteins to the telomere results in the chromosome end being packaged in a particular manner. As can be shown, this inhibits the telomerase. As the telomere shortens, this inhibition decreases, thereby providing for a telomere homeostasis. However, this homeostasis very probably has another important function: it couples telomere regulation to the system for controlling the cell cycle. This latter system is activated by way of a p53-dependent mechanism when DNA breaks or naked DNA ends are present. In aging, telomerase-negative somatic cells, the telomeres are gradually eroded as are, consequently, the opportunities for TRF and related proteins to bind as well. There are experimental grounds to indicate that, when the length falls below a given minimum, the p53-dependent checkpoint system is activated such that the cell cycle is stopped at the G1/S transition. The cell has arrived at what is termed the Hayflick senescence limit.

This point can be passed by infecting cells with cancer-inducing viruses. SV40 is an example of such a virus. This virus expresses what is termed the large tumor antigen, TAg, which binds to the tumor suppressor proteins p53 and pRB, thereby inactivating them. This leads to a defect in the checkpoint system. As a result, it is possible for a cell to divide beyond the Hayflick limit. The cell then has an extended lifespan. However, the resulting cell population is not yet immortal, that is has still not been immortalized, since there is still a second control point: this control point is termed crisis and arises as a result of the further disappearance of the telomeres. When the telomere length is approximately 2.5 kb or less, the chromosome end becomes unstable. The cell recombination apparatus is possibly also involved in this. The genetic instability is lethal for the very great majority of cells. In very rare cases, i.e. less than 1 per 10 million, a cell escapes this crisis and enters once again into replicative life. Such a cell is immortalized and consequently a potential cancer cell.

In more than 90% of cases, immortalized cells and tumor cells express the telomerase catalytic subunit. This is limiting, whereas the template RNA and TP1 appear to be expressed in most cells. By contrast, most somatic cells are negative for the telomerase catalytic subunit. Activated T and B lymphocytes, CD34-positive stem cells and mitotically active keratinocytes are exceptions to this rule. However, it has been found that, while the telomerase activity which can be measured in the cells is at best able to retard telomere loss, it cannot stop it. On the other hand, some human tumors have also been found which do not possess telomerase activity. Since these tumors frequently exhibit particularly long telomeres, it is assumed that there are alternative mechanisms for redressing telomere loss.

In order to immortalize cells, e.g. primary fibroblasts, which are already dividing, it is sufficient to add the telomerase catalytic subunit. Resting and terminally differentiated cells (e.g. adult heart muscle cells, neurons) additionally require gene functions for overcoming the cell cycle arrest. Viral oncogenes such as SV40 TAg, HPV E6 and E7, and adenovirus E1A and E1B, can be used for this purpose. However, cellular oncogenes, such as ras, myc, src, etc., can also provide the necessary growth signals.

However, the inherent problem in any immortalization is that, by accumulating mutations, these cells can develop further to become cancer cells. For this reason, it is necessary to be able to make the immortalization reversible.

In order to solve this problem, DE 100 19 195, which has not been previously published, proposes a reversible immortalization which is based on introducing a “survive gene complex” into organ-related cells. Inter alia, this gene complex contains the human telomerase catalytic subunit as well as the TAg. The complex is flanked by Lox/P sequences. The cells are propagated ex vivo for as long as required using the immortalizing property of the complex. Before transplantation into patients, the Cre recombinase is used to excise the survive complex between the Lox/P sequences. In order to be used in humans, this technique requires a guarantee that the immortalizing functions are completely removed from every cell.

According to DE 100 19 195, this is effected by combining the Cre/Lox system with the HSV thymidine kinase (TK) negative selection system. All the cells in which the survive gene complex is still functional are killed by the activity of the TK when the prodrug ganciclovir is added to the cells. A disadvantage of this technology can be seen in the fact that the survive gene complex is administered in the form of an expressible DNA sequence which can integrate randomly into the genome. It cannot be ruled out, therefore, that the DNA sequences which are distal to the LoxP sites remain in the genome even after the Cre recombinase has been successfully used.

VP22- and (HIV)TAT-fusion proteins containing an immortalization peptide are known from WO 00/61617 (Baetge et al). Such fusion proteins deal with immortalization of target cells.

Against this background, the present invention is based on the object of providing a method by which it is possible both to immortalize cells for producing regenerative tissue and to completely remortalize the cells, and of providing suitable agents for use in the novel method.

### SUMMARY OF THE INVENTION

According to the invention, this object is achieved by means of a method for transiently immortalizing cells in which immortalizing proteins are introduced into the cells from the exterior.

In the context of the present invention, immortalizing proteins are understood, on the one hand, as being transforming proteins which, in connection with being expressed in the cell, ensure that the corresponding cell divides once again, or continues to divide beyond the Hayflick limit, as achieved, for example, by the SV40 TAg. Administering such an immortalizing protein ensures, for example, that a resting, terminally differentiated cell divides once again such that tissue for a transplant patient can be produced ex vivo from the starting cells of an organ.

On the other hand, in the context of the present invention, immortalizing proteins are also understood as being telomere proteins which, when expressed in the cell, ensure that the corresponding cell remains able to replicate without limit, or once again becomes able to replicate without limit, since telomere loss during expansion is avoided, as is achieved, for example, by the telomerase catalytic subunit. The applicant possesses a plasmid which is likewise part of the subject-matter of the present invention and encodes a human telomerase catalytic subunit which is termed hTRT^{plus}, which was deposited in the DSMZ—Deutsche Sammlung von Mikroorganismen und Zellkulturen GmbH [German Collection of Microorganisms and Cell Cultures, Inhoffenstraβe 7 B 38124 Braunschweig, GERMANY] (DSM 14569) in accordance with the Budapest treaty on Nov. 17, 2001, which carries the designation pcrscript telomerase and is transfected into E. coli HB101. The DNA sequence for the immortalizing gene hTRT^{plus} can be isolated from the plasmid. This deposit was made under the provisions of the Budapest Treaty on the International Recognition of the Deposit of Microorganisms for the Purposes of Patent Procedure and the Regulations thereunder (Budapest Treaty). This assures maintenance of a viable culture of the deposit for 30 years from date of deposit. The deposit will be made available by DSMZ under the terms of the Budapest Treaty, and subject to an agreement between Applicant and DSMZ which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC § 122 and the Commissioner's rules pursuant thereto (including 37 CFR § 1.14). Availability of the deposited strain is not to be construed as a license to practice the invention in contravention of the rights granted under the authority of any government in accordance with its patent laws.

According to the invention, the transforming and telomere proteins are now added, separately or in combination, to cells which are to be expanded until the desired quantity of tissue has been produced.

However, the transforming and telomere proteins can also be employed, using one of the methods which are still to be described below, and in the embodiment which is still to be further described, for administration to patients, in order to achieve transient stimulation of cell division in vivo (transient in vivo immortalization).

Against this background, the invention also relates to a therapeutic composition which comprises at least one immortalizing protein according to the invention.

An important advantage of the novel method is to be seen in the fact that no DNA sequences are transferred into cells, which means that integration into the cell genome cannot take place. The “immortalization” only lasts as long as the immortalizing proteins continue to be administered from the exterior. Discontinuing the supply of immortalizing proteins results in the immortalization being reversed since the immortalizing proteins which are present in the cells are continually being broken down by endogenous proteases. In the context of the present invention, this process is termed transient immortalization since it only lasts as long as the immortalizing proteins are being made available externally. For this purpose, these proteins can be secreted, for example, by feeder cells or be produced recombinantly, e.g. using the Baculovirus system or in E. coli.

The gene functions possessing immortalizing properties consequently do not act on the cells to be immortalized as an expressible DNA sequence but, instead, directly as proteins. To achieve this, the cells to be immortalized are treated with immortalizing proteins, which are transferred into the cells by means of biochemical, chemical or physical administration.

When the immortalizing proteins are administered biochemically, they are fused with protein transduction domains, ligands, e.g. peptide ligands, or single chain antibodies. To do this, the immortalizing proteins are either prepared recombinantly, e.g. in a baculovirus system or E. coli system, and added directly, as purified fusion proteins, to the target cells, that is to the organ-related cells, or expressed in feeder cells which release the immortalizing proteins into the medium. The feeder cells are cocultured with the target cells such that the immortalizing proteins pass from the feeder cells into the medium and are taken up by the organ-related cells. In this connection, the feeder cells can express different fusion proteins, with it being also possible, however, to use different feeder cells, each type of which only expresses one fusion protein.

The immortalizing proteins are administered chemically using, for example, liposomes or internalizable nanoparticles. The immortalizing proteins are prepared recombinantly, purified and introduced into the target cells using these chemical methods. It is also possible for the immortalizing proteins to be coupled chemically to a non-peptide ligand and taken up into the target cells using this ligand.

The immortalizing proteins are physically administered by means of particle bombardment, electroporation or microinjection. The immortalizing proteins are prepared recombinantly, purified and introduced into the target cells using these physical methods.

For the biochemical administration, the immortalizing proteins are provided with additional amino acids at the aminoterminus or carboxyterminus, which amino acids make it possible for the proteins to be taken up from the cell culture medium using natural transport processes. This can be achieved by producing fusions of immortalizing proteins and protein transduction domains. Proteins possessing such domains are termed “messenger” or “translocating” proteins (review in: Prochiantz, 2000 Curr. Opin. Cell Biol. 12:400-406).

Many of the messenger proteins which are known today belong to the homeoproteins (e.g. engrailed, Hoxa-5 and antennapedia). Homeoproteins are transcription factors which play an important role in development processes and are found in all metazoa as well as in plants. The transcription factors bind to the DNA using a domain, i.e. the homeodomain, which is 60 amino acids in size. The homeodomains contain three helices. In the case of the homeoprotein antennapedia, it has been discovered that amino acids 43-58 in the third helix constitute the “cellular import sequence”, i.e. CIS. The penetratin peptide family was developed from this sequence (review article in: Derossi et al., 1998 Trends Cell Biol. 8: 84-87) with penetratin 1 being the original sequence.

Both the purified penetratin 1 and fusions of penetratin 1 with heterologous proteins or peptides are taken up directly, from the extracellular space into the cytoplasm or into the nucleus, by means of an atypical process which does not include the endocytosis pathway. The precise mechanism is not yet understood. The company Q-BIOgene (Heidelberg) offers two possibilities for using penetratin: 1. penetratin 1 peptide is coupled chemically to the proteins or peptides to be imported; these fusion proteins are then added to the cells and taken up by them. 2. The Q-BIOgene transVector system is used to fuse the DNA sequence for the target protein to the DNA sequence for the penetratin; the fusion protein can then be prepared recombinantly, after transforming the vector into E. coli bacteria, and purified using a HIS tag. The recombinant fusion protein is added to target cells and taken up by them.

The company Q-BIOgene Heidelberg reports that penetratin 1 can be used successfully with proteins which can be more than 100 amino acids in size. The use of penetratin, or of peptides derived therefrom, for transporting the telomerase or the T-Ag is therefore also part of the subject-matter of the invention.

A mode of administration which is envisaged within the context of the invention is that of fusing the immortalizing proteins to the voyager protein VP22. This 38 kDa protein is the product of the herpes simplex virus (HSV) gene UL49 and is a principle structure protein of the HS virion. It exhibits the special property of intercellular transport, i.e. it is transported out of the cell in which it was synthesized and into the nuclear region of the adjacent cells, as described in the literature (Elliot and O'Hare, Cell 1997, 88: 223-233). Interestingly, fusion proteins formed from VP22, e.g. VP22-GFP (green fluorescent protein) fusion proteins, also retain this property.

The inventors of the present application have fused VP22 to the SV40 large T Ag and also generated a cell line which forms and secretes this fusion protein. The inventors have been able, for the first time, to demonstrate that fusions of proteins with VP22 not only enable the target proteins to be transported into cell lines but also into primary cells.

Against this background, the present invention also relates to a fusion protein which is composed of VP22 and an advantageous protein, preferably an immortalizing protein, also preferably for transporting the advantageous protein into a primary cell.

The generation of fusion proteins composed of VP22 and telomerase, and also the preparation of corresponding feeder cell lines, also come within the context of the invention. The feeder cells are cultured together with the cells which are to be immortalized. The immortalizing proteins are released and taken up by the cells which are to be immortalized. The feeder cells are separated spatially from the target cells by means of a chamber possessing a semipermeable membrane. Taking the chamber out of the cell culture dish interrupts the supply of the immortalizing proteins; the target cells are once again mortal and in their original state.

However, aside from VP22 and penetratin, there are a number of other proteins or peptides which possess the ability to penetrate into target cells (see Table 1). The immortalizing proteins can be fused to one or more of these proteins, or to sequences from these proteins, without departing from the scope of the invention. It will also be understood that protein transduction sequences which are not listed in Tab. 1, and also protein transduction sequences which are at present not yet known, can be fused to the immortalizing proteins.

TABLE 1
Messenger proteins

| | | Location |
|---|---|---|
| Peptide/Protein | Origin | in the cell |
| FGF-1 and FGF-2 | humans, inter alia | nucleus |
| lactoferrin | humans, inter alia | nucleus |
| VP22 | herpes simplex virus | nucleus |
| TAT | human immunodeficiency virus | nucleus |
| Engrailed | humans, inter alia | nucleus |
| Hoxa-5 | humans, inter alia | nucleus |
| antennapedia homeodomain | Drosophila | nucleus |
| peptide (“penetratin”) | | |

Thus, according to a further embodiment of the invention such transduction sequences generally refer to Cell Penetrating Peptides (CPP) or Protein Transduction domains (PTD). Thus, examples of “messenger proteins” include, but are not limited to, a transport polypeptide sequence from human immunodeficiency virus (HIV) REV, a homeodomain from the Antennapedia polypeptide (“Antp HD”) or Penetratin, Engrailed or Hoxa-5, a polymer of L-arginine or D-arginine amino acid residues (“Arg repeats”), a polymer of L-lysine or D-lysine amino acid residues (“Lys repeats”), transcription factors like BETA2/neuro D, PDX-1 (“transcription factors”), any nuclear localization signal (“NLS”) like NLS derived from SV40, Histone derived peptides and other, a polymer of cationic macromolecules (“cationic polymer”); FGF-1 and FGF-2, Lactoferrin or homologues or fragments thereof.

The transduction sequences HIV REV protein is described in Suzuki et al. (Suzuki et al. 2002 J. Biol. Chem. 277:2437-2443 and Futaki 2002 Int. J. Pharmaceut. 245:1-7). Also included are the homeodomain sequence from Antennapedia (Antp HD, described, e.g., in PCT Publications WO97/12912 and WO99/11809) and sequences of Penetratin (Derossi et al. 1998 Trends Cell Biol. 8:84-87), Engrailed (Gherbassi, D. & Simon, H. H. J. 2006 Neural Transm. Suppl 47-55 Morgan, R. 2006 FEBS Lett. 580:2531-2533, Han, K. et al. 2000 Mol. Cells. 10:728-732 or Hoxa-5 (Chatelin et al. 1996 Mech. Dev. 55:111-117 and sequences containing Arg repeats (described, e.g., in Canadian Patent No. 2,094,658; U.S. Pat. No. 4,701,521; PCT Publication WO98/52614) or Lys repeats (Mai et al. 2002 J. Biol. Chem. 277:30208-30218, Park et al. 2002 Mol. Cells. 13:202-208, Mi et al. 2000 Mol. Ther. 2:339-347). Also included are the transcription factors like BETA2/neuro D, PDX-1 (Noguchi and Matsumoto 2006 Acta Med. Okayama 60:1-11, Noguchi et al. 2003 Diabetes 52:1732-1737, Noguchi et al. 2005 Biochem. Biophys. Res. Commun. 332:68-74), any nuclear localization signal (“NLS”) like NLS derived from SV40, (Yoneda et al. 1992 Exp. Cell Res. 201:313-320). Histone derived peptides (Lundberg and Johansson 2002 Biochem. Biophys. Res. Comm. 291:367-371.

Moreover, the invention refers to immortalizing proteins or polypeptides including, but are not limited to, the 12S and 13S products of the adenovirus E1A genes, SV40 small T antigen and SV40 large T antigen (and subfragments and truncated versions thereof), including the small and large T antigens (subfragments and truncated versions) of other polyomaviruses such as JK-virus and BC-virus, papilloma viruses E6 and E7, in particular E6 and E7 derived from human papillomavirus (HPV), the Epstein-Barr Virus (EBV), Epstein-Barr nuclear antigen-2 (EBNA2), human T-cell leukemia virus-1 (HTLV-1), HTLV-1 tax, Herpesvirus Saimiri (HVS), mutant p53, and the proteins from oncogenes such as myc, c-jun, c-ras, c-Ha-ras, h-ras, v-src, c-fgr, myb, c-myc, n-myc, and Mdm2, Bmi-1, E2F3, twist and cyclins such as cyclin E and D, cyclin-dependent kinases such as cdk 2, 4, 6, members of the E2F transcription factor family and growth factors known to increase proliferative activity of cells such as EGF and FGF and anti-apoptotic proteins like bcl-2, Mutants of p16/INK4a, p14/ARF, p19/ARF, p21, p27, family of pRB (retinoblastoma) proteins, ATM/ATR, Bax, Ets and PARP, in particular PARP comprising a mutation in the caspase 3 cleavage site (“uncleavable PARP”)

According to the invention the term immortalizing proteins or polypeptides refers to functional cellular proteins like, but not limited to, (proto-)onkogenes such as myc, c-jun, c-ras, c-Ha-ras, h-ras, v-src, c-fgr, myb, c-myc, n-myc, and Mdm2, Bmi-1, E2F3, twist and cyclins such as cyclin E and D, cyclin-dependent kinases such as cdk 2, 4, 6, members of the E2F transcription factor family and growth factors known to increase proliferative activity of cells such as EGF and FGF and anti-apoptotic proteins like bcl-2, wherein such functional cellular proteins or polypeptides promote the cell cycle and allow escape from senescence or apoptosis and lead to cell immortalization. According to the invention the term anti-apoptotic proteins shall mean those proteins whose presence in cells allow escape from apopotosis. Examples for this are Bcl-2 and other anti-apoptotic members of the Bcl family, survivin, anti-apoptotic virus proteins such as Epstein-Barr virus LMP1 and BHRF1 proteins. Anti-apoptotic proteins according to the invention extend to mutants of pro-apoptotic proteins such as mutant pro-apoptotic members of the Bcl familiy, mutant Bax, mutant caspases, mutant protein kinases important for death receptor signalling, mutant death receptor (e.g. mutant CD95/FAS or mutant TNF receptor). Moreover, such “anti-apoptotic proteins” refer to such proteins as disclosed in Zhivotovsky et al (Zhivotovsky and Orrenius, 2006 Carcinogenesis 27:1939-1945, Chan and Yu 2004 Clin. Exp. Pharmacol. Physiol 31:119-128, Georgiev et al. 2006 Curr. Pharm. Des. 12:2911-2921, Jarpe et al. 1998 Oncogene 17:1475-1482, Kawanishi 1996 Nippon Rinsho 54:1848-1854 (1996)) which is hereby incorporated in reference.

According to the invention the term immortalizing proteins or polypeptides refers to mutant cellular proteins or polypeptides like, but not limited to, mutants of p16/INK4a, p14/ARF, p19/ARF, p21, p27, p53, family of pRB (retinoblastoma) proteins, ATM/ATR, Bax, Ets and PARP, in particular PARP comprising a mutation in the caspase 3 cleavage site (“uncleavable PARP”). Such mutants are generated in such a way that they compete with their wild type or native protein counterparts in a dominant negative way causing loss of function of their wild type counterparts (cf. Sheppard, 1994 Am. J. Respir. Cell Mol. Biol. 11: 1-6).

Hence, such functional cellular proteins or polypeptides and/or mutant cellular proteins or polypeptides according to the invention prevent senescence or apoptosis and causing a prolonged or increased replicative lifespan of cells and leading to cell immortalization.

Thus, in a further preferred embodiment the immortalizing proteins encompass functional cellular proteins or polypeptides and/or mutant cellular proteins or polypeptides, wherein at least one functional cellular protein or polypeptide and/or mutant cellular protein or polypeptide is part of a translocation fusion polypeptide.

In another preferred embodiment of the invention the immortalization proteins are E6 or E7 derived from human papilloma virus (HPV), wherein the so called high risk HPV types (cf. TABLE 1) are preferred. Particularly preferred are E6 and E7 proteins derived from HPV 16 and HPV 18. Moreover, the E6 and E7 proteins from the so called low risk HPV types are preferred (cf. TABLE 2). Hereto particularly preferred are E6 and E7 proteins derived from HPV 6 and HPV 11. Advantageously, HPV E6 proteins inactivate the p53-dependent cell cycle control and hereby contributing to the cell cycle maintenance. Moreover, the HPV E7 proteins inactivate the retinoblastoma protein (pRB) dependent cell cycle control and hereby contributing to the cell cycle induction and maintenance. The cell cycle stimulatory effects of the E6 and E7 proteins of the high risk HPVs might be more effective than those of the low risk HPVs. However, infections with low risk HPVs are not associated with malignant transformations. Thus, the use of the E6 and E7 proteins of low risk HPVs for the purpose of transient cell immortalization is even safer than using E6 and E7 from high risk HPV types. In a further aspect of the invention, immortalization proteins of high risk and low risk HPVs can be mixed, e.g. E6 from HPV 16 together with E7 of HPV 11. In another aspect of the invention protein domains of different HPV types can be fused in order to generate a chimeric protein. For instance, a carboxy terminal domain of E6 from HPV 16 can be fused to the aminoterminal domain of E6 from HPV11, or even domains of E6 proteins can be fused with domains of E7 proteins in order to generate chimeric proteins. Of course, the said immortalization proteins can be truncated, i.e. certain peptide sequences are removed or deleted, or immortalization proteins can be modified with respect to their amino acid sequence. The aim is to use optimized immortalization proteins stimulating cell proliferation effectively but having minimal or no effects on other cell functions, namely differentiation or transformation.

TABLE 2
Classification of high- and low risk HPV types

| Classification | No of HPV type |
|---|---|
| High-risk HPV types | HPV 16, 18, 31, 33, 35, 39, 45, 51, 52, 56, 58, |
| | 59, 68, 73 and 82 |
| Low-risk HPV types | HPV 6, 11 40, 42, 43, 44, 54, 61, 70, 72 and 81 |

Another possibility of administering immortalizing proteins which is envisaged within the context of the invention is that of fusing these proteins to a receptor ligand or to a recombinant single chain antibody which is able to bind to a receptor. The RGD motif, which is found in adhesion molecules such as vitronectin, collagen and laminin, as well as in the capsid proteins of many viruses such as Coxsackie virus A9 and adenovirus, is a prototype of a ligand which can be used universally. The RGD motif contains the amino acids Arg-Gly-Asp and mediates binding to integrins, i.e. heterodimeric membrane glycoproteins which are expressed by virtually all cell types. Viruses can use this mechanism to penetrate into cells. In Exp. Nephrol. 1999, 2:193-199, Hart describes using RGD ligands for transferring molecules into cells. Within the context of the invention, the RGD motif is fused to the immortalizing proteins telomerase and TAg. These fusion proteins can be used within the context of secreting them from cocultured feeder cells or as fusion proteins which are prepared recombinantly in a baculovirus or E. coli system and which are then added directly to the cells to be immortalized. It will be understood that other ligands, incl. single chain antibodies, can also be fused or (chemically) coupled to the immortalizing proteins without departing from the scope of the invention. The description contains an implementation example of using phage display for identifying peptide ligands.

Another strategy for administering the immortalizing proteins which is envisaged in the context of the invention is that of using antibodies, in particular bispecific antibodies. Arndt et al. (Blood 1999 94:2562-2568) describe the use of a recombinant bispecific monoclonal antibody which, at one end, binds to the natural killer cell CD16 antigen and, at the other end, recognizes the human Hodgkin tumor CD30 antigen. Using the “diabody” brings about the lysis of the tumor cells by the natural killer cells. The company Affimed Therapeutics AG, Heidelberg, offers the development of special bispecific antibodies as a service. In the context of the invention, it is possible to use bispecific antibodies which, at the one end, bind the telomerase or TAg immortalizing protein, which has previously been prepared recombinantly, and, at the other end, bind to a cellular receptor and thereby bring about internalization of the immortalizing proteins.

Chemical administration makes use, for example, of cationic lipids which, for a relatively long time now, have been used for introducing nucleic acids (plasmids, vectors, ribozymes, etc.) into cells by way of forming liposomes. In J. Biol. Chem. 2001, 37: 35103-35110, Zelphati et al. describe, for the first time, using the new trifluoroacetylated lipopolyamine TFA-DODAPL together with the dioleoyl phosphatidylethanolamine DOPE. This cationic formulation, which is marketed by the company Gene Therapy Systems Inc. (10190 Telesis Court, San Diego, Calif. 92121, USA) under the trade name BioPorter, can be used to introduce peptides and proteins into cells with a high degree of efficiency. Within the context of the invention, it is possible to use the BioPorter reagent to introduce the immortalizing proteins telomerase and SV40 T-Ag, which have previously been prepared recombinantly, into the primary cells which are to be immortalized. It will be understood that it is also possible to use other suitable liposomal reagents without departing from the scope of the invention.

In recent years, the use of nanoparticles for medically administering therapeutic substances has been increasingly promoted. While nanoparticles are used, like liposomes, as carriers for therapeutic substances, they have the advantage of enclosing substances in a substantially more stable manner. Soppimath et al. (Journal of Controlled Release 2001, 70:1-20) describe the preparation and use of biodegradable nanoparticles composed of poly(D,L-lactide) (PLA), poly(D,L-glycolide) (PLG), poly(lactide-co-glycolide) (PLGA), poly(cyanoacrylate) (PCA) and poly(e-caprolactone) (PCL). Nanoparticles have a size of 10-1000 nm and can be used for packaging DNA, RNA and proteins/peptides.

Within the context of the invention, use is made of internalizable nanoparticles which gradually release the immortalizing proteins telomerase and T-Ag, which have previously been prepared recombinantly, in the cell. For special applications, it may be of importance to modify the surface of the nanoparticles so that they can bind to internalizable receptors. This can be achieved, for example, by covalently or noncovalently binding ligands or recombinant single chain antibodies (monospecific or bispecific) to the surface of the nanoparticles. Other modifications for improving the attachment of the nanoparticles to cells and their uptake into cells are possible without departing from the scope of the invention. It is also possible to carry out an electroporation for the purpose of improving the uptake of the nanoparticles into cells.

Biodegradable nanoparticles, containing immortalizing proteins which are packaged therein, are also particularly suitable for being administered in vivo, within the context of a therapy or prophylaxis, to a patient in order to bring about in vivo regeneration, for example of the heart.

Physical administration makes use, for example, of electroporation, which has been used in eukaryotic and prokaryotic cells for many years as a very good transfection means for ensuring the uptake of DNA. The cells are exposed, for a few milliseconds, to an electric field of some 100 volts, and of up to 10 000 volts in the case of bacteria. This appears to make the cell membranes porous for a short period such that even very polar macromolecules, such as DNA or RNA, can be efficiently taken up by the cells.

Lambert et al. (Biochem. Cell Biol. 1990, 4:729-734) and Morgan and Day (Methods Mol. Biol. 1995, 48:63-71) describe protocols which also enable proteins to be taken up efficiently into cells using electroporation. Within the context of the invention, electroporation can be used to transfer the immortalizing proteins, which have previously been prepared recombinantly, into the primary cells which are to be immortalized.

The microinjection of macromolecules, such as DNA, RNA and proteins, has likewise been used for many years. In this method, a stereomicroscope and a micromanipulator can be used to puncture the cell directly with a glass needle. The molecule to be transferred is then directly introduced into the desired cell compartment (cytoplasm or cell nucleus) by way of the glass needle or glass cannula. This can be carried out with a very high degree of efficiency, either manually or in a computer-controlled manner. Within the context of the invention, microinjection can be used to transfer the immortalizing proteins, which have previously been prepared recombinantly, into the primary cells which are to be immortalized.

Against this background, the present invention furthermore relates to a method for obtaining cells using the steps of: providing organ-related cells, transiently immortalizing the organ-related cells by externally supplying immortalizing proteins which are used in accordance with the invention, expanding the immortalized cells and remortalizing the expanded cells by terminating the supply of immortalizing proteins. Organ-related cells which can be used in this context are multipotent stem cells, preferably mesenchymal stroma cells or else resting, terminally differentiated starting cells of the organ, preferably cardiac muscle cells.

As a result of the transient immortalization in accordance with the invention, the cells which are prepared in this way are clinically safe. In addition, the cells can be prepared in unlimited numbers.

When multipotent stem cells are used as organ-related cells, the transiently immortalized stem cells are only expanded after at least one differentiating substance, which promotes differentiation of the stem cells into organ-specific cells, has been added. These differentiated cells are then transiently immortalized using the method according to the invention.

If, on the other hand, terminally differentiated starting cells are used, they are preferably also immortalized in connection with the transient transformation such that they can be expanded in a virtually unlimited manner.

If cells which are differentiated but still able to divide, such as neonatal cardiac muscle cells, are used, it is then only immortalization with telomere proteins, and not any transformation, which is envisaged.

The cells which have been prepared in this way can, for example, be transplanted into a cardiac infarction area, thereby, at one and the same time, substantially diminishing the risk of congestive heart failure and the risk of a secondary, fatal cardiac infarction. The method is also suitable for obtaining regenerative bone cells and cartilage cells which can be used in connection with bone trauma and cartilage trauma and in connection with chronic bone degeneration (osteoporosis). The method can also be used to prepare liver parenchyma cells for liver regeneration as well as dopaminergic cells for treating Parkinson's disease.

The method according to the invention makes it possible to produce any arbitrary quantities of primary cells for fabricating tissue extracorporeally. Endothelial cells or smooth muscle cells which have been produced using the novel method can be established on a matrix, preferably a biomatrix, for example composed of collagen or fibronectin, in order to generate heart valves or venous valves.

When, for example, producing muscle cells, preferably cardiac muscle cells, and also bone cells, preference is given to adding a differentiating substance, which is selected from the group: dexamethasone, 5′-azacytidine, trichostatin A, all-trans retinoic acid and amphotericin B, before the transiently immortalized stem cells are expanded. In this connection, it is particularly preferred if at least two, preferably four, of these differentiating substances are used prior to the transient immortalization.

Although differentiation of stem cells into cardiac muscle cells is induced by adding 5′-azacytidine, the differentiation can be improved by adding at least one additional differentiating substance. In this connection, a combination of 5′-azacytidine and trichostatin A is particularly suitable, with the inventors of the present application having found that these compounds act synergistically. The differentiation can be further optimized by additionally adding all-trans retinoic acid and amphotericin B.

Aside from the synergistic effect which lies in combining several differentiating substances, combining these substances has the further advantage that the mutagenic effect which the inventors have found 5′-azacytidine to possess is substantially reduced or even abolished.

It is thereby possible for the cardiac muscle cells, which have been obtained from stem cells in this way, to be safely used clinically.

By adding the differentiating substance dexamethasone, the stem cells are differentiated into bone cells or cartilage cells prior to the transient immortalization. In this case, too, a synergistic effect can be achieved by additionally adding the differentiating substances 5′-azacytidine, trichostatin A, all-trans retinoic acid and amphotericin B.

With regard to the differentiating substance dexamethasone, it may also be mentioned that Conget and Minguell “Phenotypical and functional properties of human bone marrow mesenchymal progenitor cells”, J. Cell Physiol. 181:67-73 have already reported that osteogenic cells can be obtained from mesenchymal stroma cells by treating with dexamethasone.

Both autologous and allogenic cells can be used in this connection as organ-related cells.

While the advantage of autologous cells lies in the immunotolerance, the allogenic cells enjoy the advantage that they are available in unlimited numbers more or less at any time.

It is then consequently possible to initially use transplantable cells which have been prepared from allogenic cells for treating a patient while further transplantable cells are prepared in parallel from the patient's autologous cells. If sufficient autologous transplantable cells are then available, it is only these which are still transplanted, such that the immunotolerance then no longer constitutes any problem.

Cells which have been prepared using the novel method, and, where appropriate, using the novel means, are likewise part of the subject-matter of the present invention. According to the invention, these cells can be used for preparing a transplant for the regeneration of an organ or for treating chronic diseases.

Against this background, the present invention also relates to a transplant which contains the cells which have been prepared in accordance with the invention.

The present invention furthermore relates to the use of the cells for regenerating an organ.

The invention also relates to the immortalizing proteins which are used in accordance with the invention, and also to nucleic acid molecules and plasmids which encode the immortalizing proteins, for expressing the immortalizing proteins, and also to cells which are transformed for expressing the immortalizing proteins, in particular feeder cells.

In particular, the invention relates to the plasmids having the designations pCMV-VP22-TAg and pcDNA-TAg-VP22, which were deposited under the deposition numbers DSM 14570 and 14568 in the DSMZ—Deutsche Sammlung von Mikroorganismen und Zellkulturen GmbH (German Collection of Microorganisms and Cell Cultures, Inhoffenstraβe 7 B 38124 Braunschweig, GERMANY) in accordance with the Budapest treaty, on Nov. 17, 2001, which plasmids are transfected into E. coli HB101 and can be used to prepare the fusion proteins. These deposits were made under the provisions of the Budapest Treaty on the International Recognition of the Deposit of Microorganisms for the Purposes of Patent Procedure and the Regulations thereunder (Budapest Treaty). This assures maintenance of a viable culture of the deposits for 30 years from date of deposit. The deposits will be made available by DSMZ under the terms of the Budapest Treaty, and subject to an agreement between Applicant and DSMZ which assures permanent and unrestricted availability of the progeny of the cultures of the deposits to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC § 122 and the Commissioner's rules pursuant thereto (including 37 CFR § 1.14). Availability of the deposited strain is not to be construed as a license to practice the invention in contravention of the rights granted under the authority of any government in accordance with its patent laws.

Finally, the invention relates to a kit for transient immortalization, which kit contains the plasmids according to the invention and/or immortalizing proteins.

In addition to the plasmids, nucleic acid molecules and/or immortalizing proteins, the kit according to the invention can contain the substances and materials which are additionally required for a biochemical, chemical or physical administration.

The kit can then be used to transiently immortalize and expand allogenic or autologous donor cells before they are then transplanted for the purpose of organ regeneration.

Other advantages ensue from the description and the enclosed drawing.

It will be understood that the abovementioned features, and those which are still to be explained below, can be used not only in the combinations which are in each case indicated but also in other combinations, or on their own, without departing from the scope of the present invention.

### BRIEF DESCRIPTION OF THE DRAWINGS

The invention is now explained with the aid of implementation examples and the enclosed drawing. In the drawing:

FIG. 1 shows the detection, by Western blotting, of TAg in the fusion protein. The indicated plasmids were transiently transfected into T antigen-negative 10SW cells (IntroGene). 48 hours after transfection, protein extracts were obtained, the proteins were fractionated on SDS-page, blotted onto PVDF membranes and detected using monoclonal anti-SV40 T antigen antibodies. The second antibody was peroxidase-conjugated; the ECL technique was used to detect the signals. The prominent band in lanes 1 and 6 is the TAg. The arrow indicates the position of the fusion protein.

FIG. 2 shows the detection, by Western blotting of VP22 in the fusion protein. The extracts were obtained, and subjected to further treatment, as described in the legend to FIG. 1. The first antibody was an antiserum directed against VP22 while the second antibody was peroxidase-conjugated as in FIG. 1. The arrow indicates the position of the fusion protein.

FIG. 3 shows cell stainings which show the generation of VP22-TAg-expressing cell lines. Human 10SW cells were stably transfected with the plasmid pCMV-VP22-TAg. This resulted in cell line 10SW-22T, which is a mixed population comprising cells which are expressing fusion protein and cells which are not expressing the protein. A and D, staining of all the cells with the DNA-intercalating substance DAPI; B, staining of the cells with TAg antibodies; C, DAPI and TAg double staining; E, staining of the cells with VP22 antibodies; F, double staining with DAPI and VP22. As a result of fusing the TAg to the voyager protein VP22, the protein diffuses into adjacent untransfected cells. In C and F, the technique of double staining shows producer cells containing fusion protein in the cell nucleus and cytoplasm (horizontal arrows), and cells which, after importation, only contain the fusion protein in the cell nucleus (vertical arrows). NB: the VP22 antibody is considerably more sensitive than the TAg antibody; considerably more positive cells are therefore seen. However, there are also some cells in F which do not appear to be positive, that is which do not appear either to express or import the fusion protein. Either the effective concentration of the fusion protein is too low for the detection in these cases or there are some cells within this cell population which are not competent to import protein. This is possibly associated with particular cell cycle phases.

FIG. 4 shows cell stainings which show VP22 being imported into primary human cardiac muscle cells. CO60 hamster cells were infected with an adenovirus for expressing the VP22 (Ad-CMV-VP22). Two days later, the infected cells were cocultured with primary human cardiac muscle cells. A, staining all the cells with DAPI; B, using immunofluorescence to detect the presence of VP22; V, using a special marker to identify the CO60 cells; D, double staining of VP22 and CO60 marker. The horizontal arrow indicates a doubly stained cell, which is consequently a CO60 cell which contains VP22. The two vertical arrows indicate cardiac muscle cells which have imported VP22.

FIG. 5 shows the map of plasmid pCMV-VP22-TAg. Description: vector for expressing the gene fusion VP22-Tag under control of the CMV promoter, starting vector is pVP22 from Invitrogen (Groningen, The Netherlands). Elements: CMV-promoter: position 209-863; Gene fusion from VP22-Tag: position 911-3985; Bovine growth hormone polyadenylation signal (BGHpA): position 4182-4409

FIG. 6 shows the sequence of the VP22-TAg gene fusion (SEQ ID NO: 29) illustrated in FIG. 5;

FIG. 7 shows the map of plasmid pcDNA-TAg-VP22. Description: vector for expressing the Tag-VP22 gene fusion under the control of the CMV promoter; the starting vector is pcDNA3.1 from Invitrogen (Groningen, The Netherlands). Elements: CMV promoter: position 232-819; VP22-Tag gene fusion: position 932-3997; bovine growth hormone polyadenylation signal (BHGpA): position 4107-4331.

FIG. 8 shows the sequence of the TAg-VP22 gene fusion (SEQ ID NO: 30) illustrated in FIG. 7;

FIG. 9 shows the map of plasmid pCRscript-telomerase. Description: vector for cloning the human telomerase catalytic subunit; the starting vector is pCRscript from Stratagene (Heidelberg). Elements: telomerase variant: position 714-4257 (contains 104 base pair intron from position 933 to position 1037.

FIG. 10 shows the sequence of the telomerase gene hTRT^{plus} (SEQ ID NO: 31) illustrated in FIG. 9.

### DETAILED DESCRIPTION OF THE PREFERRED EMBODIMENT

### Example 1

### Cloning Fusion Proteins Composed of SV40-Tag and VP22

Expression constructs which enable both VP22-T-antigen and T-antigen-VP22, i.e. both N-terminal and C-terminal fusion proteins, to be expressed were prepared.

a. Mutagenesis

The first thing that was done in this regard was to use site-directed PCR mutagenesis to prepare a plasmid which contained the SV40 T-antigen without any stop codon (primers, see Table 3). This plasmid was named pIND-TAg (-stop). The SV40 TAg was obtained from Prof. W. Deppert Heinrich Pette Institut für Experimentelle Virologie und Immunologie der Universität Hamburg [Heinrich Pette Institute for Experimental Virology and Immunology at Hamburg University]. A kit supplied by Stratagene Inc. was used for the site-directed mutagenesis.

TABLE 3
Primers for the site-directed mutagenesis
Primers

| | |
|---|---|
| 5′-cctccccctgaacctgaaacaagatctgaatgc | (SEQ ID NO: 1) |
| aattgttgttgttaacg-3′ | |
| 5′-cgttaacaacaacaattgcattcaggatcttgt | (SEQ ID NO: 2) |
| ttcaggttcagggggagg-3′ | |

b. Clonings

A stop codon-free T antigen fragment was obtained from plasmid pIND-TAg (-stop) by subjecting it to double digestion with the restriction endonucleases EcoRI and BglII.

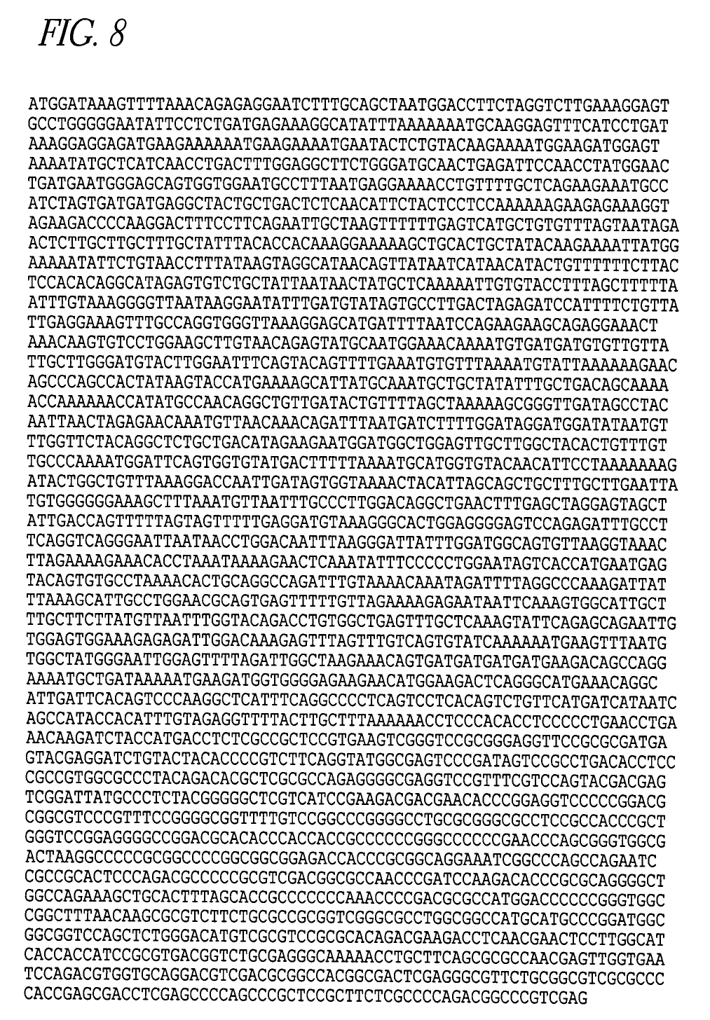

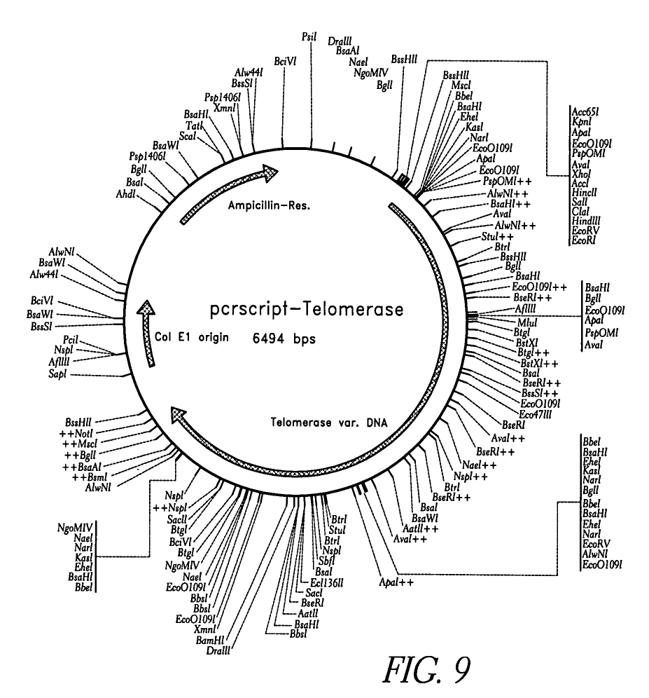

The VP22 fragment was prepared by digestion with NotI and BgII from the plasmid pCDTK49, and, after having been digested with NotI and EcoRI, pcDNA3.1 (Invitrogen) was used as the vector. This resulted in the expression construct pcDNA-TAg-VP22, which carries a CMV promoter-regulated cassette for expressing the TAg-VP22 fusion protein (see plasmid map and sequence of pcDNA-TAg-VP22 in FIGS. 7 and 8). The plasmid was deposited, under the receipt number DSM 14568, in the DSMZ [German Collection of Microorganisms and Cell Cultures], in accordance with Budapest treaty, on Nov. 17, 2001; it is transfected into E. coli HB101.

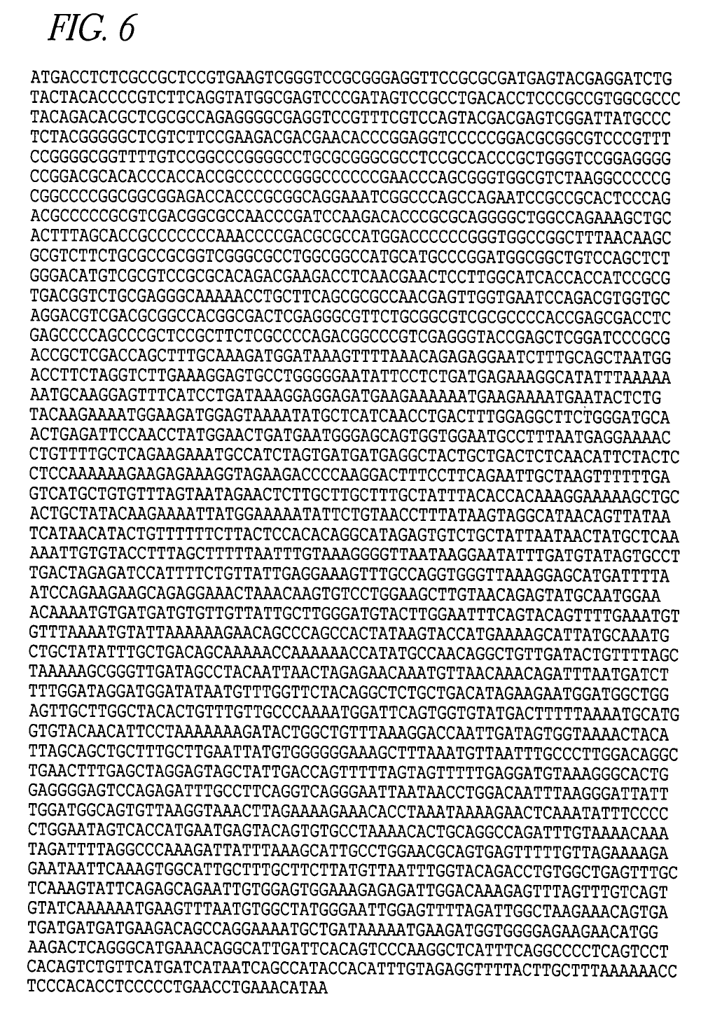

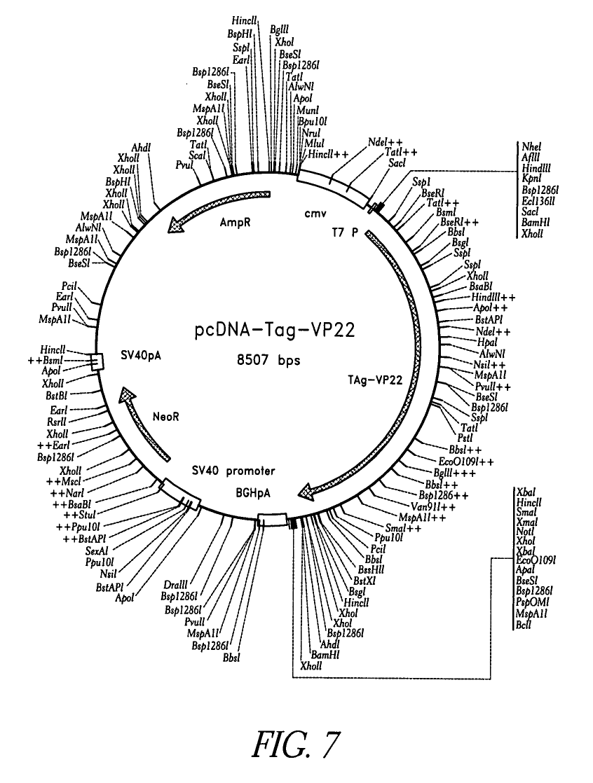

The expression plasmid pCMV-VP22-TAg was prepared using the vector pVP22 (Invitrogen). To do this, the T antigen fragment was obtained from pIND-TAg, by subjecting the latter to double digestion with KpnI and EcoRI, and ligated into the pVP22 vector, which had likewise been opened with KpnI and EcoRI. (See plasmid map and sequence of pcCMV-VP22-TAg in FIGS. 5 and 6). The plasmid was deposited, under the receipt number DSM 14570, in the DSMZ [German Collection of Microorganisms and Cell Cultures], in accordance with Budapest treaty, on Nov. 17, 2001; it is transfected into E. coli HB101.

The fusion protein constitutes a fusion of the VP22 protein to the N-terminus of the large T antigen; the expression cassette is likewise regulated by the CMV promoter.

### Example 2

### Demonstrating the Fusion Protein Composed of SV40 Tag and VP22

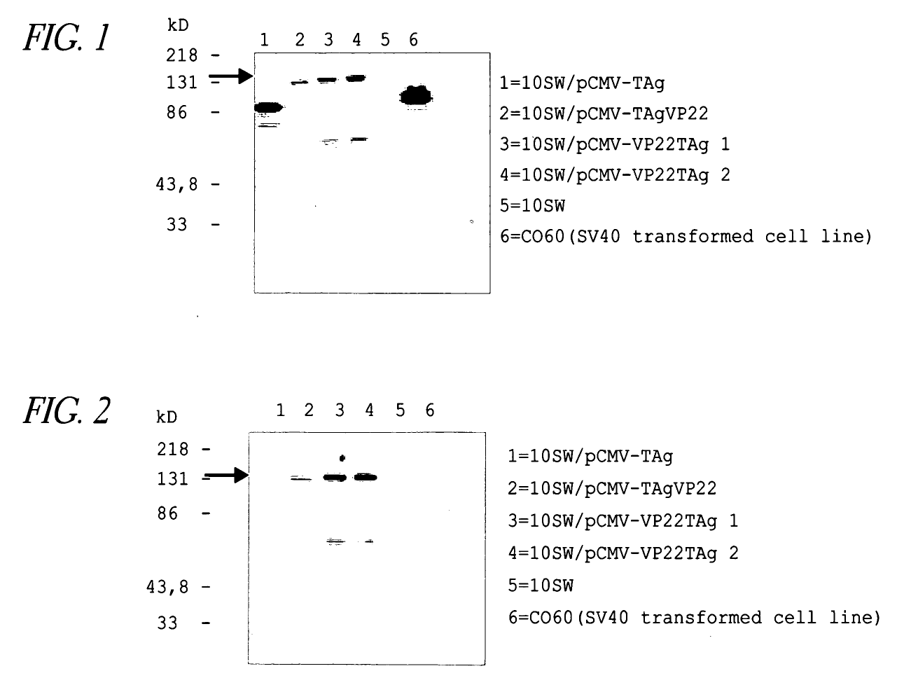

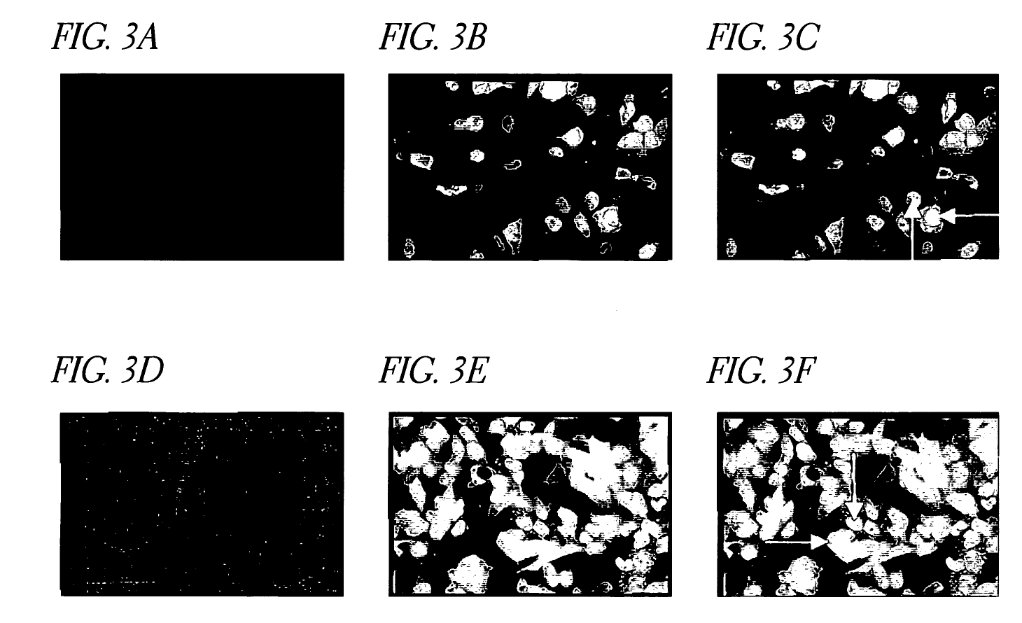

The fact that the clonings had indeed produced fusion proteins composed of VP22 and SV40 large T antigen was demonstrated in transfection experiments which were followed by Western blot analyses (FIGS. 1 and 2).

In the first place, transient transfection was used to introduce the new expression constructs into T antigen-negative cells. After that, protein extracts were obtained from the cells, with these extracts being fractionated in SDS polyacrylamide gels and the protein being blotted onto PVDF membranes and analyzed using both monoclonal anti-SV40 T antigen antibodies and a polyclonal anti-VP22 antiserum.

It was found that the cells which had been transfected with the fusion constructs expressed proteins which were of the size of the expected fusion proteins and were also recognized by both types of antibody.

### Example 3

### Generating Feeder Cell Lines

In order to generate a cell line which can function as a “fusion protein producer”, 10SW cells (human retina cells transformed with adenoviruses E1A and E1B) were transfected with the fusion protein-expressing plasmids and then selected with G418, since the expression constructs also mediate a resistance to neomycin.

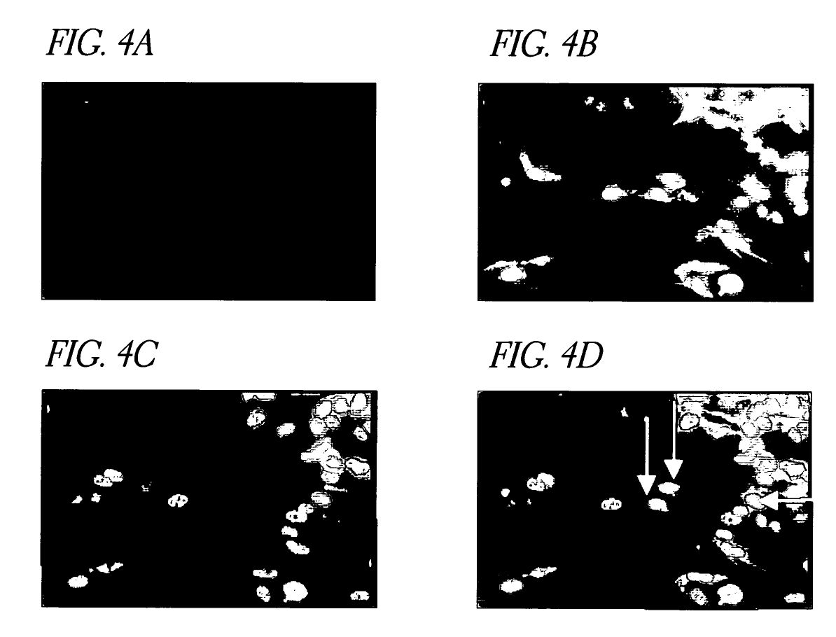

After a selection lasting several weeks, a mixed population of G418-resistant cells was produced. Immunohistochemical analyzes showed that, as was to be expected, not all the cells expressed the fusion protein (see FIG. 3). The functionality of the system was demonstrated using in-situ localization techniques (immunofluorescence, double staining). Most of the cells of the feeder cell line produce the fusion protein, which diffuses into neighboring cells. This can be recognized from the fact that, while producing and excreting cells contain the fusion protein both in the cytoplasm and in the cell nucleus, importing cells only contain the fusion protein in the cell nucleus (FIG. 3, C and F). The cells are subcloned by end-point dilution such that feeder cell lines which are homogeneous, i.e. expressing the immortalizing protein in each cell, are obtained. These feeder cells were then used, in coculture experiments, to investigate the export of the TAg-VP22 fusion protein into primary cells, and likewise to investigate the functionality of the TAg in an NIH3T3 transformation assay. In addition, coimmunoprecipitation was used to investigate the binding of the TAg to p53 and pRB.

Both mortal and immortalized cells can be used as feeder cells.

### Example 4

### Importation of VP22 Fusion Proteins into Primary Cells

The inventors demonstrated in the following way that the VP22 protein also transports proteins which are fused to it into primary cells. This was previously only known in the case of tumor cell lines. Thus, an investigation was carried out to determine whether a VP22 fusion protein is also imported into human fibroblast, human smooth muscle cells and human cardiomyocytes.

To do this, CO60 cells (an SV40-transformed hamster cell line) were infected with a recombinant adenovirus which contains a gene fusion, composed of VP22 and GFP (green fluorescent protein), under the control of the CMV promoter. The cells which were infected with this adenovirus were sown, together with in each case one type of said primary cells, on sterile cover slips. After an incubation lasting approx. 48 hours, the cover slips were fixed with formaldehyde and analyzed immunohistochemically. A combination of mouse anti-SV40 T antigen antibody and rabbit anti-VP22 antibody was used for the purpose.

It was consequently possible to identify the originally infected CO60 cells by the fact that they were positive for the SV40 T antigen and for the VP22-GFP fusion protein. Primary cells which have obtained the VP22-GFP by means of intercellular transport processes are only positive for VP22 and not for the SV40 T antigen.

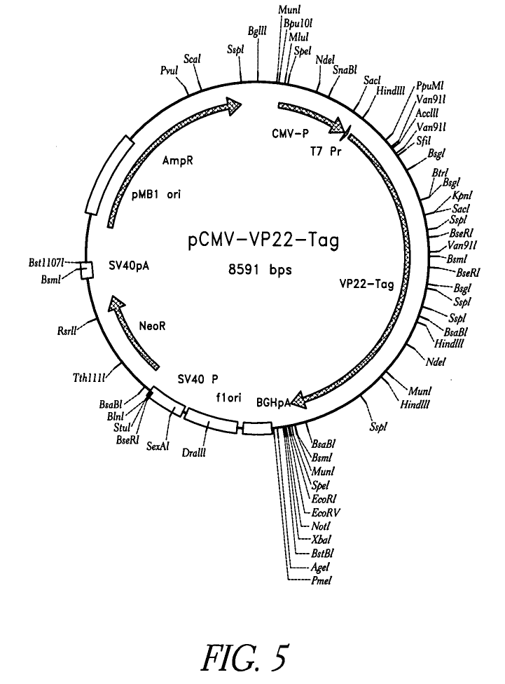

It was found that it was possible for VP22 fusion proteins to be transported not only into immortalized cells but also into the primary human cardiac muscle cells which were investigated (FIG. 4). The function of the protein which is fused to VP22, that is of the GFP or the immortalizing gene, is retained in the primary cells.

### Example 5

### Cloning the Human Telomerase Catalytic Subunit

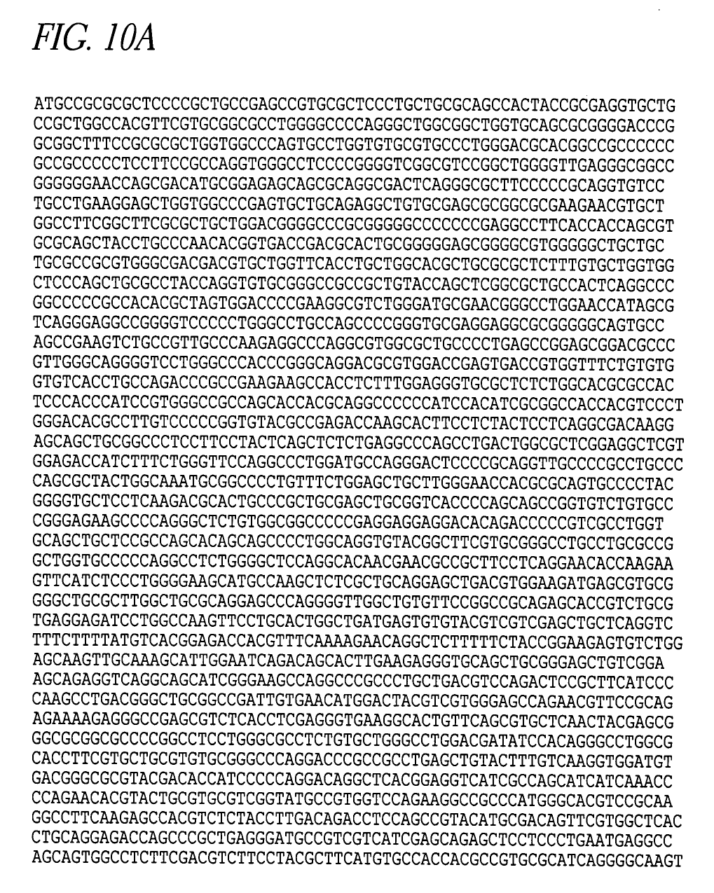

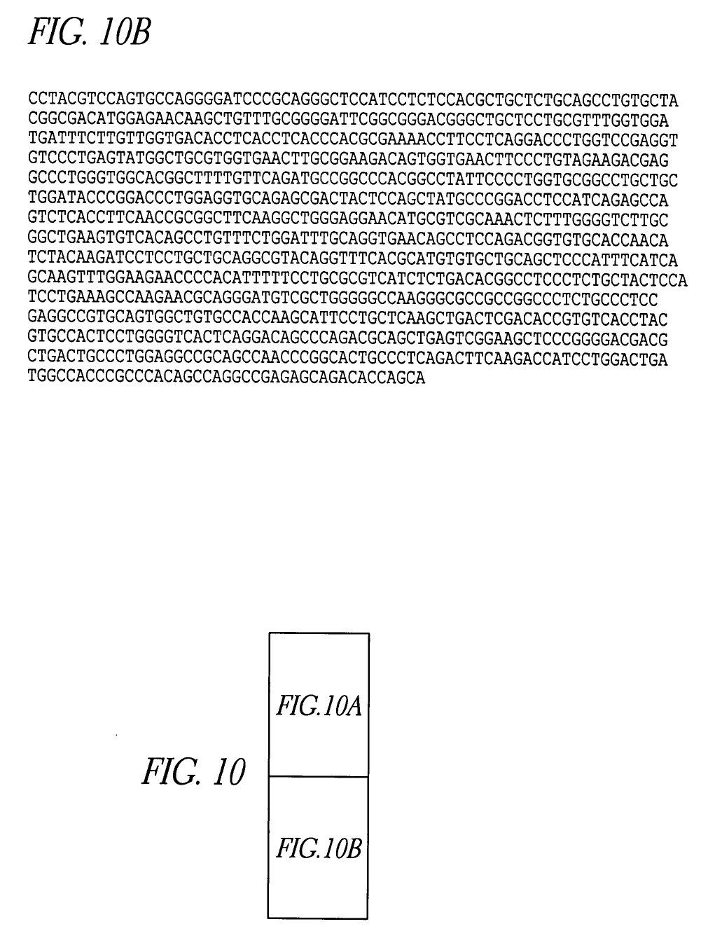

The most important gene for the immortalization, i.e. the human telomerase catalytic subunit, was cloned from human cells (the DNA sequence of this telomerase fragment, hTRT^{plus}, and the map of the plasmid containing the human telomerase catalytic subunit, are given in FIGS. 9 and 10). The telomerase cDNA has a very high G/C content and is extremely difficult to clone. Throughout the world, therefore, there are only very few groups which possess their own telomerase cDNA. In contrast to the known and published telomerase, the telomerase shown in FIGS. 9 and 10 has a 109 bp intron. Introns have a transcript-stabilizing effect. In order to differentiate it from the known telomerase, this telomerase is designated hTRT^{plus}. The telomerase was cloned in the following steps:

1. mRNA was isolated from human Jurkat cells (lymphoma T cell line).

2. This mRNA was transcribed into cDNA molecules in a reverse transcription using specific RT primers (see Table 4) (MMLV reverse transcriptase).

3. Fragments which in each case encompassed approx. 500 to 1000 bp of the coding regions of the human telomerase gene were obtained, in individual PCR reactions (see Table 5), from the resulting cDNA pool. In these reactions, the primers were chosen such that this resulted in fragments which overlapped in those regions of the telomerase cDNA which in each case contained a restriction cleavage site which was unique for this cDNA.

4. The fragment which was located furthest 5′ was not generated by reverse transcription of telomerase mRNA but, instead, obtained directly by carrying out a PCR on genomic DNA obtained from HeLa cells (human cervical carcinoma cell lines). During this, use was made of a PCR technique which was special for highly G/C rich sequences.

5. The resulting fragments (Table 6) were cloned into plasmid vectors (Table 7) and propagated in bacteria. All of the fragments were checked by sequencing. A base exchange was found at position 993 in telomerase cDNA. At this point, the C which was originally present has been replaced with an A. However, this is a silent mutation, i.e. this base exchange does not have any effect on the amino acid sequence.

In a variety of 3-component ligations, the individual fragments were joined together to form a human telomerase variant which contained the entire coding region. The corresponding plasmid is termed PCR script-telomerase (plasmid map and sequence of hTRT^{plus} in FIGS. 9 and 10) and was deposited in the DSMZ [German Collection of Microorganisms and Cell Cultures] under the deposition number DSM 14569, in accordance with Budapest treaty, on Nov. 17, 2001; it is transfected into E. coli HB101.

TABLE 4

Primers which were employed for the specific
reverse transcription of telomerase mRNA

| Primer for the reverse | |
|---|---|
| transcription | Sequence |
| RT-telo-4 | 5-CTCATATATTCAGTAT-3 |
| | (SEQ ID NO: 3) |
| RT-telo-3 | 5-CTGGACACTCGCTCA-3 |
| | (SEQ ID NO: 4) |
| RT-telo-2 | 5-TCAGCCGGACATGCA-3 |
| | (SEQ ID NO: 5) |
| RT-telo-1 | 5-TCACTCAGGCCTCAG-3 |
| | (SEQ ID NO: 6) |

TABLE 5
Primers employed for obtaining overlapping PCR fragments

| | | |
|---|---|---|
| | | Rest |
| riction - | | |
| cleavage | | |
| Pcr Primer | Sequence | site |

| | | | |
|---|---|---|---|
| PCR-telo-10 | 5′-GCTGGTGTCTGCTCTCG-3′ | (SEQ ID NO: 7) | — |
| PCR-telo-11 | 5′-CTGCAGCAGGAGGATCTTGTAGATG-3′ | (SEQ ID NO: 8) | ApaLI |
| PCR-telo-6 | 5′-GCAGGTGAACAGCCTCCAGAC-3′ | (SEQ ID NO: 9) | ApaLI |
| PCR-telo-R13 | 5′-CACAGGCTGCAGAGCAGCGTGGAG-3′ | (SEQ ID NO: 10) | BamHI |
| PCR-telo-F12 | 5′-GTCCTACGTCCAGTGCCAGGGGATC-3′ | (SEQ ID NO: 11) | BamHI |
| PCR-telo-R15 | 5′-GAGCACGCTGAACCAGTGCCTTCAC-3′ | (SEQ ID NO: 12) | XhoI |
| PCR-telo-F14 | 5′-AGAGGGCCGAGCGTCTCACCTCGA-3′ | (SEQ ID NO: 13) | XhoI |
| PCR-telo-R17 | 5′-CGCTCATCTTCCACGTCAGCTCCTGC-3′ | (SEQ ID NO: 14) | SphI |
| PCR-telo-F16 | 5′-CTCAGGAACACCAAGAAGTTCATC-3′ | (SEQ ID NO: 15) | SphI |
| PCR-telo-R19 | 5′-CCTGGCATCCAGGGCCTGGAACCCA-3′ | (SEQ ID NO: 16) | BssS-I |
| PCR-telo-F18 | 5′-TCCCTACTCAGCTCTCTGAGGCCCAGC-3′ | (SEQ ID NO: 17) | BssSI |
| PCR-telo-F20 | 5′-TTGCTGGTGGCTCCCAGCTGCGCCTAGGA-3′ | (SEQ ID NO: 18) | SexAI |
| PCR-telo-R21 | 5′-AGTGGCAGCGCCGAGCTGGTACAGC-3′ | (SEQ ID NO: 19) | SexAI |
| PCR-telo-7 | 5′-ATGCCGCGCGCTCCCCGCTGCCAG-3′ | (SEQ ID NO: 20) | — |

TABLE 6
PCR fragments. Fragments T2 to T6 were obtained
by the PCR amplification of telomerase cDNA: fragment
T7II was obtained by the PCR amplification of
genomic DNA and contains an intron.

| Fragment | Primers | Region | Comments | Restriction cleavage sites |
|---|---|---|---|---|
| T2 | F12, 10 | 2524bp- | 3′-region + | 5′-terminal: BamHI |
| | | 3494bp | stop codon | 3′-terminal: — |
| T3 | R13, F14 | 2001bp- | — | 5′-terminal: XhoI |
| | | 2589bp | | 3′-terminal: BamHI |
| T4 | R15, F16 | 1517bp- | — | 5′-terminal: SphI |
| | | 2051bp | | 3′-terminal: XhoI |
| T5 | R17, F18 | 1088bp- | — | 5′-terminal: BssSI (BsiI) |
| | | 1596bp | | 3′-terminal: SphI |
| T6 | R19, F20 | 530bp- | — | 5′-terminal: SexAI |
| | | 1179bp | | 3′-terminal: BssSI (BsiI) |
| T7II | 7, R19 | 55bp- | Start codon | 5′-terminal: — |
| | | 1176bp | | 3′-terminal: BssSI (BsiI) |

TABLE 7
Intermediate constructs, each containing one fragment

| Vector | Insert |
|---|---|
| pIND-T21 | T2 |
| pIND-T3 | T3 |
| pIND-T4 | T4 |
| pIND-T5 | T5 |
| pIND-T6 | T6 |
| pCRII-T7II | T7II |

### Example 6

### Cloning a Vector for Expressing a Fusion Protein, Consisting of VP22 and the Telomerase Catalytic Subunit, in Mammalian Cells

A vector was constructed, with the vector enabling a fusion protein consisting of VP22 and the telomerase catalytic subunit to be expressed in mammalian cells, and with the VP22 sequences being located 5′ of the telomerase sequences.

a. Mutagenesis of the Pcrscript-Telomerase Construct

In order to clone the expression vector, a stop codon located upstream of the start codon in the telomerase sequence was removed from the pcrscript-telomerase construct by means of site-directed PCR mutagenesis (kit supplied by Stratagene) and a Kpn I cleavage site was inserted in its place. The primer #1 listed in Table 8 was used for this purpose. Following PCR using an appropriate reverse primer, the plasmid which resulted from this was used for a second site-directed PCR mutagenesis. This mutagenesis, which was carried out using primer #2, served to remove the stop codon which was located at the 3′ end of the telomerase sequence while at the same time introducing an Age I cleavage site, thereby making possible an in-frame fusion with the His tag which was present in the vector pVP22/myc-His (Invitrogen).

b. Cloning the pCMV-VP22-Telo-His construct

In order to clone the expression construct pCMV-VP22-Telo-His, the restriction endonucleases Kpn I and Age I were used to excise a fragment from the mutagenized plasmid, with this fragment containing the telomerase sequences, possessing a start codon located at the 5′ end but lacking the stop codon at the 3′ end. This fragment was then cloned directionally into the Invitrogen pVP22/myc-His vector, which had been opened with Kpn I and Age I, such that a gene fusion consisting of N-terminal VP22, telomerase and C-terminal His Tag, under the control of the CMV promoter, was obtained.

TABLE 8

Primers for site-directed PCR mutagenesis for
generating the stop codon-free telomerase
fragment. The letters in bold give the
sequences of the newly inserted restriction
cleavage sites Kpn I (Primer #) and Age I
(primer #2), respectively.

| | |
|---|---|
| Primer | Sequence |

| | |
|---|---|
| Primer #1 | 5′-aagcttgatatcgaattcgggtaccatgccgcgcgctc |
| | cccgctcccgg-3′ |
| | (SEQ ID NO: 21) |
| Primer #2 | 5′-gacttcaagaccatcctggacaccggtccacccgccca |
| | cagccaggccgag-3′ |
| | (SEQ ID NO: 22) |

### Example 7

### Generating a Feeder Cell Line for VP22 Telomerase

A VP22-telemorase-expressing feeder cell line was prepared in analogy with the TAg-VP22 feeder cell line by stably transfecting the pCMV-VP22-Telo-His construct into 10SW cells. Since the construct contains a gene for resistance to neomycin, it was possible to select for the cells which were stably expressing the VP22-telomerase protein by adding G418.

### Example 8

### Coculturing Feeder Cells with Primary Cells

The company Nunc GmbH, Wiesbaden, supplies special cell and tissue culture inserts which enable feeder cells to be cocultured with the primary cells which are to be immortalized.

The membranes of the Nunc inserts are intended for the attachment and proliferation of adherent cells. While one cell type (e.g. feeder cells) can be cultured on the membrane, another cell type (e.g. primary cells) can be kept in the bottom of the well in the appropriate multidish without the two cultures coming into direct contact with each other. On the other hand, ions, proteins and other substances can diffuse freely through the pores of the membrane. Furthermore, the size of the substances which pass through can be specified by the choice of various pore sizes.

In the context of the invention, the feeder cells described in the preceding examples are sown on these membrane inserts. For the industrial scale, the sizes of the cell culture chambers, and of the membrane inserts which are suitable for them, are adapted appropriately.

It is also possible, within the context of the invention, to use bioreactors for the mass culture of both the feeder cells and the primary cells. Feeder cells and primary cells are sown in two separate chambers within the bioreactor. The chambers are separated by a semipermeable membrane such that the immortalizing proteins are able to diffuse to the primary cells.

The feeder cells can also be present, together with the primary cells, in a mixed culture. In this case, it is appropriate to use the suspension cells as feeder cells and to use monolayer cells as primary cells, or vice versa. This thereby makes it possible to separate the feeder cells from the primary cells mechanically after immortalization has taken place. The feeder cells can also be killed by stably transfecting a cytotoxic gene (e.g. an expressible HSV thymidine kinase) selectively after adding the appropriate prodrug (in this case ganciclovir).

Other techniques for separating feeder cells and primary cells are also possible (e.g. using surface molecules for sorting with a fluorescence activated cell sorter [FACS] or magnetic activated cell sorter [MACS]).

### Example 9

### Cloning a Baculoviral Construct for Obtaining Purified Recombinant Fusion Protein Consisting of VP22 and the Telomerase Catalytic Subunit

The VP22-telomerase fusion protein is obtained in a baculo expression system which, on the one hand, enables the protein to be secreted in the cell, and consequently enables it to be folded and modified in an native manner, and, on the other hand, enables the protein to be purified by affinity chromatography.

a. Mutagenesis of the Plasmids pCMV-VP22-Telo-His and pMelBac(A)

The starting constructs for the cloning are the plasmid “pCMV-VP22-Telo-His” and the baculoviral expression vector pMelBac(A) (Invitrogen), in which an additional Hind III restriction cleavage site is in each case inserted by means of site-directed PCR mutagenesis (kit supplied by Stratagene). In the case of the pCMV-VP22-Telo-His plasmid, the primer #1 listed in Table 9 is used for this purpose, with this primer inserting a further Hind III cleavage site 3′ of the His tag, in addition to the Hind III cleavage site which is located between the CMV promoter and the VP22 sequence. In the case of the pMelBac vector, the mutagenesis using primer #2 (Table 9) inserts an additional Hind III cleavage site into the multiple cloning site directly downstream of the secretion signal.

TABLE 9

Site-directed PCR mutagenesis primers for
inserting additional Hind III cleavage sites
(bold letters) into the constructs
pCMV-VP22-Telo-His (primer #1) and pMelBac(A)
(primer #2), respectively.

| | |
|---|---|
| Primer | Sequence |

| | |
|---|---|
| Primer #1 | 5′-caccattgagtttaaacccgcaagcttgcctcgactgt |
| | gccttctagttgc-3′ |
| | (SEQ ID NO: 23) |
| Primer #2 | 5′-tacatttcttacatctatgcgaagctttggggatccga |
| | gctcgagatctgc-3′ |
| | (SEQ ID NO: 24) |

b. Cloning the Construct pMelBac-VP22-Telo-His

In order to clone the secreting baculo expression vector, restriction digestion with Hind III is used to excise the telomerase-containing fragment from the mutagenized construct pCMV-VP22-Telo-His, with this fragment being isolated and then cloned into the mutagenized vector pMelBac(A), which had likewise been cleaved with Hind III. The clones (pMelBac-VP22-Telo-His) in which the honey-bee melittin secretion signal present in pMelBac is located N-terminally in-frame with the VP22-telomerase-His fragment are identified and analyzed by restriction cleavage and sequencing.

### Example 10

### Cloning a Baculoviral Construct for Obtaining Purified Recombinant Fusion Protein Consisting of Vp22 and The Sv40 T Antigen

The recombinant VP22-Tag fusion protein is obtained, in analogy with the VP22-telomerase fusion protein, in a secreting baculo expression system. The starting constructs for cloning the baculoviral expression vector are the plasmid pCMV-VP22-TAg and the baculoviral expression vector pMelBac(A) (Invitrogen).

a. Mutagenizing the pCMV-VP22-TAg Construct

Site-directed PCR mutagenesis (kit supplied by Stratagene) employing primer #1 (Table 10) is initially used to insert an additional Age 1 cleavage site into the pCMV-VP22-TAg construct downstream of the SV40 T antigen sequence while at the same time removing the stop codon which is present in that position. A second site-directed PCR mutagenesis, employing primer #2 (Table 10), is then used to insert a Bgl II cleavage site between the CMV promoter and VP22 protein sequences, with the Hind III cleavage site which is present at that position being lost.

TABLE 10

Primers for site-directed PCT mutagenesis in
the vector pCMV-VP22-Tag, for inserting an
Age I cleavage site downstream of the SV40
T antigen sequence (primer #1) and a Bgl II
cleavage site upstream of the start codon for
the VP22 sequences (primer #2), respectively.
The letters in bold specify the sequences of
the newly inserted restriction cleavage site
Age I (primer #1) and Bgl II (primer #2),
respectively.

| | |
|---|---|
| Primer | Sequence |

| | |
|---|---|
| Primer #1 | 5′-acacctccccctgaacctgaaacaaccggtgaatgcaa |
| | ttgttgttgttaacgggga-3′ |
| | (SEQ ID NO: 25) |
| Primer #2 | 5′-ggagacccaagctggctagttaagagatctatgacctc |
| | tcgccgctccgtgaagtcg-3′ |
| | (SEQ ID NO: 26) |

b. Modifying the Vector pMelBac(A)

The two restriction cleavage sites Bgl II and Pme I are inserted into the vector pMelBac(A) 3′ of the honey-bee melittin secretion sequence by opening the vector with Bam HI and cloning in a double-stranded oligonucleotide which contains the two restriction cleavage sites. The sequences of the two complementary single strands of the oligonucleotide, which are hybridized with each other prior to insertion into the pMelBac vector, are given in Table 11.

TABLE 11

The single-stranded deoxyoligonucleotides
designated oligo #1 and oligo #2 are hybridized
with each other prior to be cloned in the
vector pMelBac(A), with overhanging ends of the
Bam HI cleavage site being formed at each end.

| | |
|---|---|
| Oligo | Sequence |

| | | |
|---|---|---|
| Oligo #1 | 5′-gatccagatctgtttaaacg-3′ | (SEQ ID NO: 27) |
| Oligo #2 | 5′-gatccgtttaaacagatctg-3′ | (SEQ ID NO: 28) |

c. Cloning the Baculoviral Expression Vector pMelBac-VP22-Tag-His

By excising an Age I fragment from the mutagenized pCMV-VP22-Tag construct and then religating, the sequence for the SV40 T antigen is brought into the immediate vicinity of the His tag which is present in the construct such that the VP22-Tag fusion protein which is to be expressed is provided C-terminally with the His tag. After that, the restriction endonucleases Bgl II and Pme I are used to excise a fragment from this construct, which is designated pCMV-VP22-Tag-His, with this fragment then being cloned directionally into the modified vector pMelBac, which has been cleaved with the same restriction enzymes, thereby forming the vector pMelBac-VP22-Tag-His.

### Example 11

### Obtaining and Purifying the Vp22-Telomerase-His and/or Vp22-Tag-His Fusion Proteins

The proteins are expressed and purified in accordance with Invitrogen's instructions, with the newly constructed vectors pMelBac-VP22-Tag-His and pMelBac-VP22-Telo-His being used for the purpose.

### Example 12

Identifying Peptide and Antibody Ligands for Transferring Immortalizing Proteins into Cardiomyocytes

Random peptide phage display libraries supplied by New England Biolabs are used to identify peptide ligands which interact with, and are internalized by, the cardiomyocytes which are to be immortalized. These libraries contain 7mer or 12mer peptides which are fused to the P3 protein of filamentous phages. The phages are incubated with the cells in the presence of chloroquine, with the addition of the chloroquine preventing phages which are internalized in lysosomes from being degraded. After non-binding phages have been washed away, and surface-associated phages have been detached from the cells by altering the pH, the internalized phages are released by lyzing the cells. This affinity selection is repeated several times (panning). After that, the relevant parts of the phage DNA are sequenced and competitive ELISA using appropriately labeled synthetic peptides is used to check the binding affinity and internalization rates of the corresponding peptide motifs.

In order to identify single-chain antibodies which are likewise to be used for transferring the above-described proteins, single-chain phagemid libraries are employed, instead of the random peptide phage display libraries, for the affinity selection.

### Example 13

### Preparing Organ-Related Cells

Multipotent stem cells which, prior to the transient immortalization and expansion, still have to be differentiated into organ-specific cells, or else already differentiated starting cells from the given organ, can be used as organ-related cells.

In addition, it is necessary to distinguish between autologous cells from the given patient and allogenic cells from a donor.

Bone marrow mesenchymal stroma cells are used as stem cells. These stroma cells are able to differentiate into osteoblasts, myoblasts, adipocytes and other cell types. In hospitals, bone marrow is routinely obtained, under operating theater conditions, for allogenic bone marrow transplantation. However, only the hematopoietic stem cells are required in this connection whereas the mesenchymal stem cells, which are of interest in this present case, are obtained as a byproduct.

On the other hand, mesenchymal stem cells can also be obtained from peripheral blood.

The stem cells which have been obtained in this way are sown in conventional cell culture dishes and cultured in alpha-MEM or IDEM medium containing 10% FCS as well as antibiotics such as penicillin, streptomycin or amphotericin B.

Liver hepatocytes are set up directly as a primary culture. Dopaminergic starting cells are removed within the context of an organ donation. Cardiac muscle cells can be obtained for the immortalization both as stem cells from bone marrow and as starting cells within the context of a cardiac muscle biopsy.

### Example 14

### Expansion and Differentiation

Starting cells which are already terminally differentiated are expanded in a customary medium in the added presence of immortalizing proteins or in coculture with feeder cells without any further steps being required.

However, if the cells to be immortalized are cells which are capable of replication, for example mesenchymal stem cells from the bone marrow, it is first of all necessary, by adding differentiating substances, to obtain differentiation into the organ-specific cells before the transient immortalization can take place.

For example, by treating them with 5′-azacytidine, mesenchymal stem cells can be differentiated into cardiomyogenic cells; Makino et al. 1999 J. Clin. Invest. 103:697-705. Treating stem cells which have the developmental potential of cardiac muscle cells with 5′-azacytidine induces differentiation processes by means of demethylation. This very probably activates the promoter of essential cardiac muscle differentiation genes which are still unknown.

However, the inventors have found that 5′-azacytidine has mutagenic potential. For this reason, and in accordance with the invention, the differentiation of stem cells into cardiac muscle cells is improved by adding at least one further differentiating substance. The substance trichostatin A (TSA) is envisaged for this purpose. TSA brings about inhibition of histone deacetylation. This histone deacetylation is connected to transcriptional repression of CpG methylations.

CpG islands, that is regions containing several CpG dinucleotides, are mainly present in promoters. The methylations can substantially inhibit the activity of a CpG-rich promoter. This takes place, for example, when 5′-azacytidine is incorporated into the DNA of replicating cells since, because of the aza group at position 5, no methylation due to cellular processes can take place at this position.

When combined, 5′-azacytidine and TSA can consequently act synergistically as has already been demonstrated in tumor cells; see Cameron et al. Nat. Genet. 21:103-107.

According to the invention, this synergy is applied to the differentiation of stem cells into cardiac muscle cells. The differentiation is further optimized by additionally adding all-trans retinoic acid and amphotericin B. Retinoic acid is a differentiating substance which, in the myoblast cell line H9C2, favors a cardiac muscle phenotype as against a skeletal muscle phenotype; see Menard et al. J. Biol. Chem. 274: 29063-29070.

Amphotericin B is also able to favorably influence differentiation in the direction of cardiac muscle cells; see Phinney et al. J. Cell. Biochem. 72:570-585.

The advantage of using a combination of several differentiating substances is that this achieves synergistic effects which substantially reduce, or even abolish, the mutagenic effect of 5′-azacytidine. This is of crucial importance for the subsequent clinical use of the stem cell-derived cardiac muscle cells.

When the differentiating substance dexamethasone (Conget and Minguell J. Cell Physiol. 181:67-73) is used on its own or in combination with the four differentiating substances described above, it is possible to differentiate stem cells into bone and cartilage cells.

The cells which have been differentiated in this way are then transiently immortalized for further expansion.

### Example 15

### Transplantation

After remortalization and appropriate quality control have been effected, conventional techniques are used to transplant the cells into the damaged organs by, for example, using a syringe to inject them into the organ. This can be done repeatedly since, as a result of the immortalization, material is available in any desired quantity.

Without the immortalization, a stem cell could only give rise to approx. 5×10⁸ to 1×10⁹ cells, or, in the case of a relatively old donor, possibly even only 1×10⁶ cells. This would probably be inadequate for regeneration. Furthermore, in the case of autologous transplantation, the patient has to wait until the cells have replicated to provide the requisite number of cells. By contrast, in the case of allogenic transplantation, the desired number of cells can be provided at any time.

In special emergencies, it makes sense to first of all carry out an allogenic transplantation and then subsequently to switch to autologous transplantation.

What is claimed is:

1. A method for transiently immortalizing cells, comprising introducing immortalizing proteins into the cells from the exterior, wherein the immortalizing proteins employed are transforming proteins of at least one of viral oncogenes selected from SV40 TAg, JK-virus and BC-virus, HPV E6, HPV E7, adenovirus EIA and adenovirus EIB, the Epstein-Barr Virus (EBV), Epstein-Barr nuclear antigen-2 (EBNA2), human T-cell leukemia virus-1 (HTLV-1), HTLV-1 tax, Herpesvirus Saimiri (HVS), mutant p53 and of cellular oncogenes selected from one of myc, c-jun, c-ras, c-Ha-ras, h-ras, v-src, c-fgr, myb, c-myc, n-myc, and Mdm2, Bmi-1, E2F3, twist or cyclins such as cyclin E and D, cyclin-dependent kinases such as cdk 2, 4, 6, or members of the E2F transcription factor family and growth factors such as EGF and FGF and anti-apoptotic proteins, mutant cellular proteins; or wherein the immortalizing proteins employed are telomere proteins, thereby avoiding a telomere loss during expansion; and wherein the immortalizing proteins are fused to one of a messenger protein, receptor ligands and antibodies, thereby forming fusion proteins, wherein the messenger protein are selected from the group of human immunodeficiency virus (HIV) REV, a homeodomain from the Antennapedia polypeptide or Penetratin, Engrailed or Hoxa-5, a polymer of L-arginine or D-arginine amino acid residues, a polymer of L-lysine or D-lysine amino acid residues, transcription factors like BETA2/neuro D, PDX-1, nuclear localization signal, Histone derived peptides, a polymer of cationic macromolecules, FGF-1 and FGF-2, lactoferrin or; homologues or fragments thereof.

2. The method of claim 1 wherein the telomere proteins employed are a catalytic subunit, hTRTplus (DSM 14569), of human telomerase.

3. The method of claim 1 wherein HPV E6, HPV E7 is selected from Low risk HPV types.

4. The method of claim 3 wherein the Low risk HPV types are selected from HPV 6 and HPV 11.

5. The method of claim 1 wherein the anti-apoptotic protein is at least one of Bcl family, BcI-2, survivin, anti-apoptotic virus proteins such as Epstein-Barr virus LMP1 and BHRF1 proteins and mutant pro-apoptotic members of the Bcl familiy, mutant Bax, mutant caspases, mutant protein kinases, mutant death receptor.

6. The method of claim 1 wherein the mutant cellular protein is at least one of p16/INK4a, p14/ARF, p19/ARF, p21, p27, family of pRB (retinoblastoma) proteins, ATM/ATR, Bax, Ets and PARP.

7. The method of claim 1 wherein the immortalizing proteins are bound to an antibody at least one of bispecific antibody which binds, by way of its second specificity, to a cellular receptor, thereby bringing about internalization of the immortalizing proteins.

8. The method of claim 1 wherein the immortalizing proteins are administered in vivo by nanoparticles.

9. The method of claim 1 wherein the fusion proteins are prepared recombinantly, purified and then added to the cells which are to be immortalized transiently, or administered in vivo.

10. The method of claim 1 wherein the fusion proteins are expressed in feeder cells and released by the feeder cells into a medium in which the feeder cells are cocultured with the cells which are to be immortalized transiently.

11. The method of claim 10 wherein the feeder cells are spatially separated by a chamber possessing a semi-permeable membrane, from the cells which are to be immortalized transiently, and wherein the feeder cells are then removed from the medium for the cells to be remortalized.

12. The method of claim 10 wherein the feeder cells are stably transfected with at least one plasmid which encodes a fusion protein which is selected from the group: comprising VP22-Tag (DSM 14570), Tag-VP22 (DSM 14568), VP22-Telo and Telo-VP22, wherein Telo denotes the catalytic subunit hTRTplus (DSM 14569) of human telomerase.

13. The method of claim 6 wherein use is made of at least two types of feeder cells, of which one type secretes a fusion protein containing a transforming protein and the other type secretes a fusion protein containing a telomere protein.

14. The method of claim 1 wherein the immortalizing proteins are transported by one of liposomes and nanoparticles into the cells which are to be immortalized transiently.

15. The method of claim 1 wherein the immortalizing proteins are transported by one of electroporation and microinjection into the cells which are to be immortalized transiently.

16. A method for obtaining cells, comprising the steps of:
providing organ-related cells,
transiently immortalizing the organ-related cells by externally supplying immortalizing proteins,
expanding the immortalized cells, and
remortalizing the expanded cells by terminating the external supply of immortalizing proteins.

17. The method of claim 16 wherein the organ-related cells employed are multipotent stem cells including bone marrow mesenchymal stroma cells.

18. The method of claim 16 wherein the organ-related cells employed are one of dividing and resting, terminally differentiated starting cells of the organ, including cardiac muscle cells.

19. The method of claim 18, wherein the starting cells are transformed in connection with the immortalizing.

20. The method of claim 16 wherein the organ-related cells employed are autologous cells.

21. The method of claim 16 wherein the organ-related cells employed are allogenic cells.

22. A cell prepared by the method of claim 16.

23. The method of claim 16, further comprising the step of preparing a transplant for regenerating an organ.

24. The method of claim 23, further comprising the step of treating chronic diseases.

25. A transplant, comprising the cell of claim 23.

26. The method of claim 23, further comprising the step of regenerating an organ.

27. An immortalizing protein for use in the method of claim 1.

28. The immortalizing protein of claim 27, comprising a transforming protein adapted to overcome a cell cycle arrest of the cells.

29. The immortalizing protein of claim 1 wherein the immortalizing protein is fused to one of a messenger proteins, receptor ligands, and antibody thereby forming a fusion protein.

30. A catalytic subunit, hTRTplus, of human telomerase, as encoded by a plasmid DSM 14569.

31. A therapeutic composition for transiently immortalizing a cell in vivo comprising the immortalizing protein of claim 27.
